(19) United States
(12) Patent Application Publication
(10) Pub. No.: US 2024/0398311 A1
(43) Pub. Date: Dec. 5, 2024

(54) PSEUDOMONOPOLAR ELECTRODE CONFIGURATIONS FOR EMG SENSING

(71) Applicant: Meta Platforms Technologies, LLC, Menlo Park, CA (US)

(72) Inventors: Jonathan REID, Summit, NJ (US); Steven Joseph KOBER, New Canaan, CT (US); Brendan Patrick FLYNN, Redmond, WA (US); Tyler Henry CHRISTENSEN, Monroe, WA (US); Paul BRANCHE, New York City, NY (US); Akshay Sunil YEMBARWAR, Milpitas, CA (US)

(21) Appl. No.: 18/803,241

(22) Filed: Aug. 13, 2024

Related U.S. Application Data
(63) Continuation-in-part of application No. 17/818,511, filed on Aug. 9, 2022.
(60) Provisional application No. 63/558,027, filed on Feb. 26, 2024.
(60) Provisional application No. 63/520,035, filed on Aug. 16, 2023.
(60) Provisional application No. 63/268,232, filed on Feb. 18, 2022.
(60) Provisional application No. 63/233,170, filed on Aug. 13, 2021.

(51) Int. Cl. A61B 5/313; A61B 5/00; A61B 5/256; A61B 5/296; A61B 5/304; A61B 5/305

(52) U.S. Cl. CPC A61B 5/313; A61B 5/256; A61B 5/296; A61B 5/304; A61B 5/305; A61B 5/6824; A61B 5/6831; A61B 5/742; A61B 2560/0462

(57) ABSTRACT

According to some embodiments, a device for sensing neuromuscular signals is provided. The device may comprise a plurality of signal electrodes aligned along an interior portion of a wearable structure, each signal electrode being configured to detect neuromuscular signals. The device may comprise a plurality of amplifiers, wherein each amplifier includes (i) a first input operatively coupled to a corresponding signal electrode, (ii) an inverting input, and (iii) an output corresponding to a neuromuscular signal channel. The device may comprise one or more buffers configured to tap a voltage at the inverting input of a respective amplifier of the plurality of amplifiers. The device may comprise circuitry configured to operatively couple a plurality of outputs of the plurality of amplifiers to generate a common mode reference signal, wherein the common mode reference signal is provided to the inverting input of one or more amplifiers of the plurality of amplifiers.

### CROSS-REFERENCE TO RELATED APPLICATIONS

This application claims priority under 35 U.S.C. § 119 to U.S. Provisional Application No. 63/520,035 filed on Aug. 16, 2023 and U.S. Provisional Application No. 63/558,027 filed on Feb. 26, 2024. This application claims priority as a continuation-in-part of U.S. application Ser. No. 17/818,511, filed on Aug. 9, 2022, which claims priority to U.S. Provisional Application No. 63/268,232, filed on Feb. 18, 2022 and U.S. Provisional Application No. 63/233,170, filed on Aug. 13, 2021. The contents of each of these applications are incorporated by reference herein in their entirety for all purposes.

### BACKGROUND

Surface Electromyography (sEMG) electrodes are used to measure action potential signals as they propagate down the muscle fibers. These action potential signals can be correlated to motor cortex activity. With wearable devices (e.g., wrist-worn devices such as smart watches or fitness trackers) becoming more common, incorporating sEMG sensing into a wearable device may provide useful information, for example, to detect hand, wrist, finger, and/or arm activity. However, use of sEMG electrodes in consumer devices is difficult, for example, because consumer devices require dry electrodes placed on the skin surface. Conventional techniques for using dry surface electrodes may lead to less robust detection of sEMG activity, which can lead to poor quality data and/or inaccurate data.

### SUMMARY

Devices, systems, and techniques for pseudomonopolar electrode configurations for EMG sensing are provided.

According to some embodiments, a device for sensing neuromuscular signals is provided. The device may comprise a wearable structure configured to be worn by a user. The device may further comprise a plurality of signal electrodes aligned along an interior portion of the wearable structure configured to be proximate to a skin surface of the user when the device is donned by the user, each signal electrode of the plurality of signal electrodes being configured to detect neuromuscular signals of the user. The device may further comprise a plurality of amplifiers corresponding to the plurality of signal electrodes, wherein each amplifier includes (i) a first input operatively coupled to a corresponding signal electrode of the plurality of signals electrodes, (ii) an inverting input, and (iii) an output corresponding to a neuromuscular signal channel. The device may further comprise one or more buffers configured to tap a voltage at the inverting input of a respective amplifier of the plurality of amplifiers. The device may further comprise circuitry configured to operatively couple a plurality of outputs of the plurality of amplifiers to generate a common mode reference signal, wherein the common mode reference signal is provided to the inverting input of one or more amplifiers of the plurality of amplifiers.

In some examples, the device may further comprise a switch disposed across each buffer of the one or more buffers. In some examples, a switch associated with a first buffer of the one or more buffers causes the first buffer to be enabled, and wherein switches associated with the remaining buffers of the one or more buffers cause the remaining buffers to be disabled.

In some examples, the circuitry comprises a set of resistors operatively coupled to output of the one or more buffers configured to generate an average of the outputs of the one or more buffers. In some examples, the device further comprises a switch disposed across each resistor of the set of resistors. In some examples, the switch may be utilized to short each resistor of the set of resistors.

In some examples, a given output of a given amplifier of the plurality of amplifiers is operatively coupled to the inverting input via one or more resistors. In some examples, the device may further comprise switches disposed across at least one of the one or more resistors, wherein a given switch is configured to cause the at least one of the one or more resistors to be shorted.

In some examples, the wearable structure comprises a wrist-worn structure.

In some examples, the plurality of signal electrodes are disposed circumferentially around the interior portion of the wearable structure.

In some examples, the neuromuscular signals are associated with wrist extensor muscles and/or wrist flexor muscles.

According to some embodiments, a device for sensing neuromuscular signals is provided. The device may comprise a wearable structure configured to be worn by a user. The device may further comprise a plurality of signal electrodes aligned along an interior portion of the wearable structure configured to be proximate to a skin surface of the user, each signal electrode of the plurality of signal electrodes configured to detect neuromuscular signals. The device may further comprise a plurality of amplifiers corresponding to the plurality of signal electrodes, wherein an amplifier of the plurality of amplifiers has: a first input operatively coupled to a corresponding signal electrode of the plurality of signals electrodes; an inverting input; and an output corresponding to a neuromuscular signal channel. The device may further comprise circuitry configured to operatively couple a plurality of outputs of the plurality of amplifiers and a buffered output of at least one reference electrode to generate a common mode reference signal, wherein the common mode reference signal is provided to the inverting input of each amplifier of the plurality of amplifiers.

In some examples, the at least one reference electrode is selected from the plurality of signal electrodes. In some examples, the at least one reference electrode is selected from the plurality of signal electrodes by shorting one or more resistors that operatively couple the amplifier associated with the at least one reference electrode to the inverting input.

In some examples, the at least one reference electrode comprises two or more reference electrodes.

In some examples, the device further comprises a buffer that receives signal from the at least one reference electrode and generates the buffered output. In some examples, an output impedance of the buffer is less than about 300 ohms.

In some examples, the wearable structure comprises a wrist-worn structure.

In some examples, the plurality of signal electrodes are disposed circumferentially around the interior portion of the wearable structure.

In some examples, the neuromuscular signals are associated with wrist extensor muscles and/or wrist flexor muscles.

### BRIEF DESCRIPTION OF THE DRAWINGS

Illustrative embodiments are described in detail below with reference to the following figures.

FIG. 1 is a schematic diagram of an example electrode configuration that utilizes a single common reference electrode in accordance with some embodiments.

FIG. 2 is a schematic diagram of an example electrode configuration that utilizes multiple reference electrodes to generate a common reference signal in accordance with some embodiments.

FIG. 3A is a schematic diagram of an example electrode configuration that utilizes signal electrodes to generate a common reference signal in accordance with some embodiments.

FIG. 3B is an example of an implementation of an electrode configuration that utilizes signal electrodes to generate a common reference signal in accordance with some embodiments.

FIG. 4A is a schematic diagram of an example electrode configuration that utilizes a signal generated from signal electrodes to generate a common reference signal in accordance with some embodiments.

FIG. 4B is an example of an implementation of an electrode configuration that utilizes a signal generated from signal electrodes to generate a common reference signal in accordance with some embodiments.

FIG. 5 is a schematic diagram that illustrates generation of the common reference signal of the example electrode configuration depicted in FIG. 4A in accordance with some embodiments.

FIG. 6 is a flowchart of an example process for algorithmically determining a common reference signal in accordance with some embodiments.

FIGS. 7A-7D illustrate example electrode placements on a wrist-worn device in accordance with some embodiments.

FIG. 8 is a simplified block diagram of an example of a computing system that may be implemented as part of a mobile device and/or a user device according to certain embodiments.

FIG. 9 is an example of an implementation of an electrode configuration that generates an output generated common reference signal using one or more buffers in accordance with some embodiments.

FIGS. 10-12 illustrate example implementations of electrode configurations for which a single point reference is generated in accordance with some embodiments.

FIG. 13 illustrates an electrode configuration in which an electrode may be configured as either an input channel or an output channel in accordance with some embodiments.

FIG. 14 illustrates an electrode configuration in which an output generated common reference signal architecture may be used to implement a single point reference implementation in accordance with some embodiments.

FIG. 15 illustrates an electrode configuration in which an output generated common reference signal architecture may be used to implement a multi-point reference implementation in accordance with some embodiments.

The figures depict embodiments of the present disclosure for purposes of illustration only. One skilled in the art will readily recognize from the following description that alternative embodiments of the structures and methods illustrated may be employed without departing from the principles, or benefits touted, of this disclosure.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Surface electromyography (sEMG) is used to measure action potential signals as they propagate down the muscle fibers. These action potential signals are correlated to motor cortex activity. Thus, sEMG may be used to detect muscle activity, and, based on the detected muscle activity, infer motor cortex activity. This may be useful to, for example, infer a person's intended motion pattern. sEMG may be particularly useful when implemented on wearable devices, such as a wrist-worn device (e.g., a smart watch, a fitness tracker, etc.) or an arm-worn device. For example, sEMG activity may be used to detect finger, hand, wrist, and/or arm muscle activity, which may be used to infer the wearer's intended movement with respect to pointing, gesturing, pinching or grabbing, writing, etc. Continuing this example, an inferred intended movement may be used for interacting with user interfaces without touching a touchscreen or utilizing a stylus, mouse, or keyboard, interacting with virtual reality (VR) and/or augmented reality (AR) environments, and for many other potential use cases.

Consumer devices that utilize sEMG typically use dry surface electrodes rather than wet electrodes or penetrating electrodes, due to wearer comfort and safety concerns. While comfortable for the wearer, dry surface electrodes have disadvantages that affect the quality of the recorded sEMG data. For example, dry surface electrodes may have a higher impedance, which may decrease signal to noise ratio. As another example, conventional techniques generally utilize either monopolar electrode configurations, in which each signal electrode is referenced to a non-electrically active reference electrode, or differential sensing, in which the difference between two signal electrodes is measured. Each has drawbacks. For example, monopolar sensing signals incorporate common mode signal that is common to all electrodes. With dry surface electrodes, the common mode signal may be dominated by power line interference or other artifactual signals, which may be much larger than the signal of interest. With differential sensing, the common mode signal is subtracted out—this allows the signal of interest to be obtained (i.e., by subtracting out power line interface), there is some signal of interest in the common mode signal, which is discarded with differential sensing. For example, with respect to a wrist-worn device, the common mode signal may indicate muscle activity in muscles fibers that are not strictly longitudinal (e.g., from elbow to wrist). Moreover, differential sensing requires additional electrodes to obtain a given number of signal channels.

Described herein are electrode configurations and techniques for generating a common reference signal used by multiple signal electrodes to generate corresponding sEMG signal channels. Use of a common reference signal is generally referred to herein as a “pseudomonopolar” configuration, because, similar to a monopolar configuration, signal electrodes are referenced to a common signal (e.g., the common reference signal), which allows portions of the common mode signal to be incorporated into each of the sEMG signal channels. The signals from signal electrodes may then be amplified with respect to the common reference signal. However, unlike a true monopolar configuration, the pseudomonopolar configurations described herein may effectively discard artifactual signals in the common mode signal, such as power line interference signals. Accordingly, the pseudomonopolar configurations described herein may provide good artifact rejection while incorporating portions of the common mode signal that are useful and/or of interest, such as muscle activity in non-longitudinal fibers. This may enable for more accurate inference of intended movement patterns.

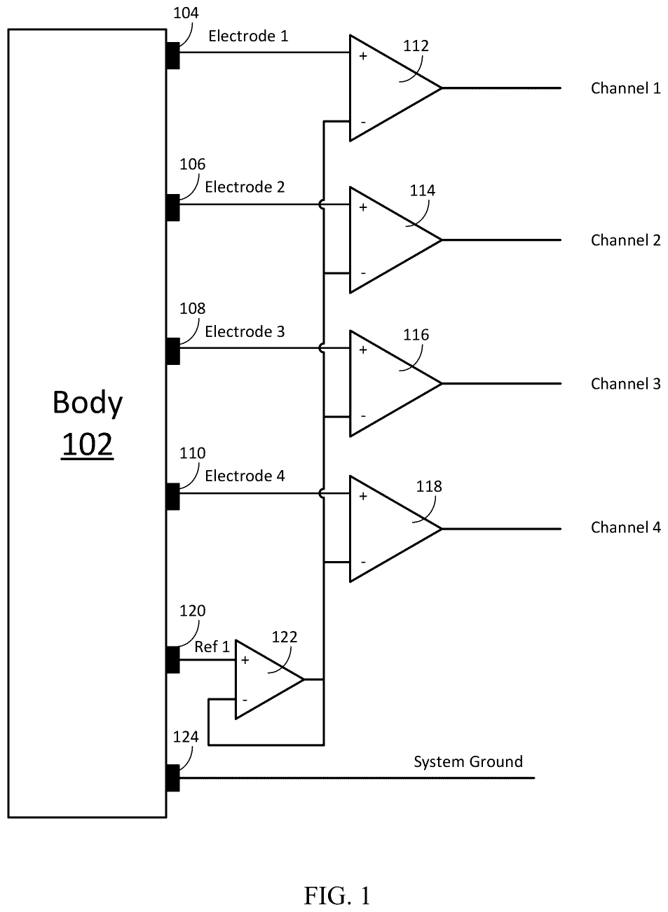

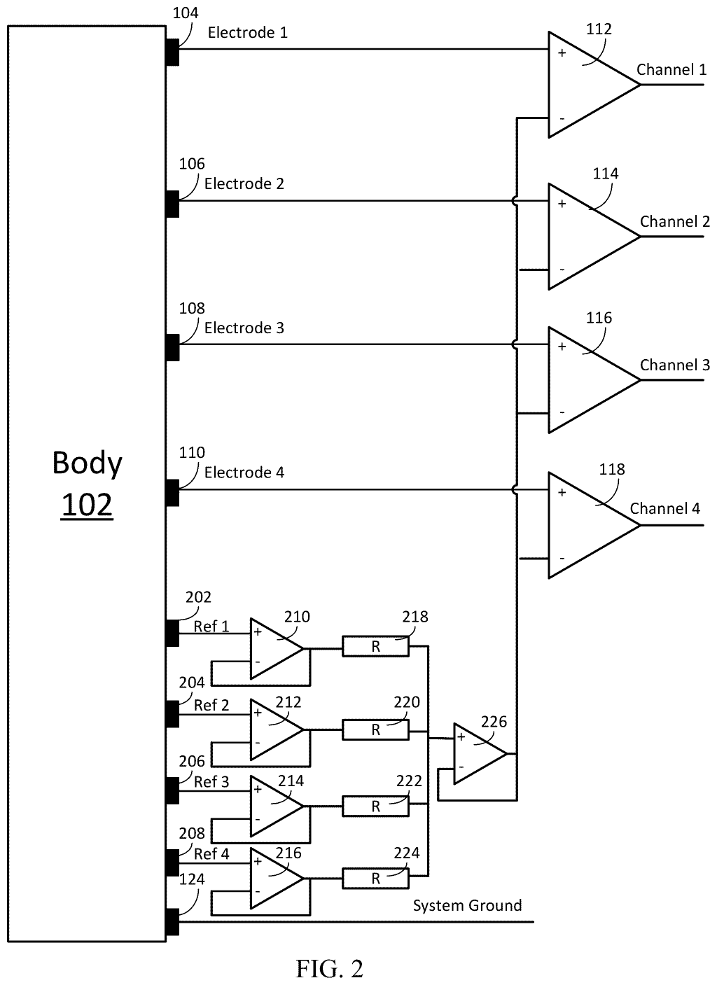

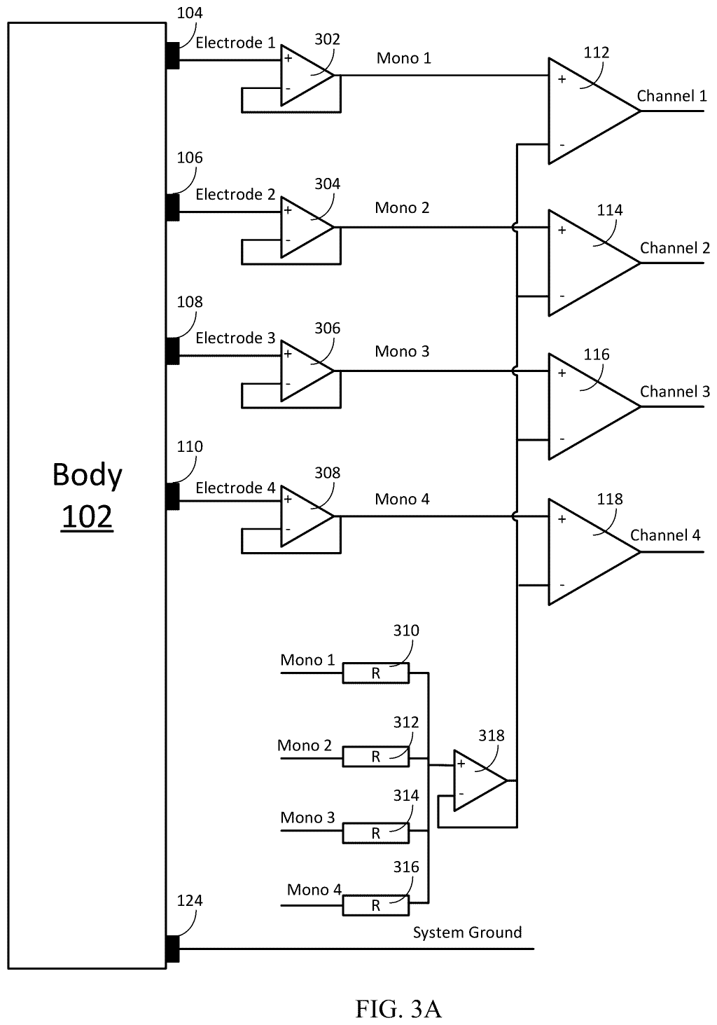

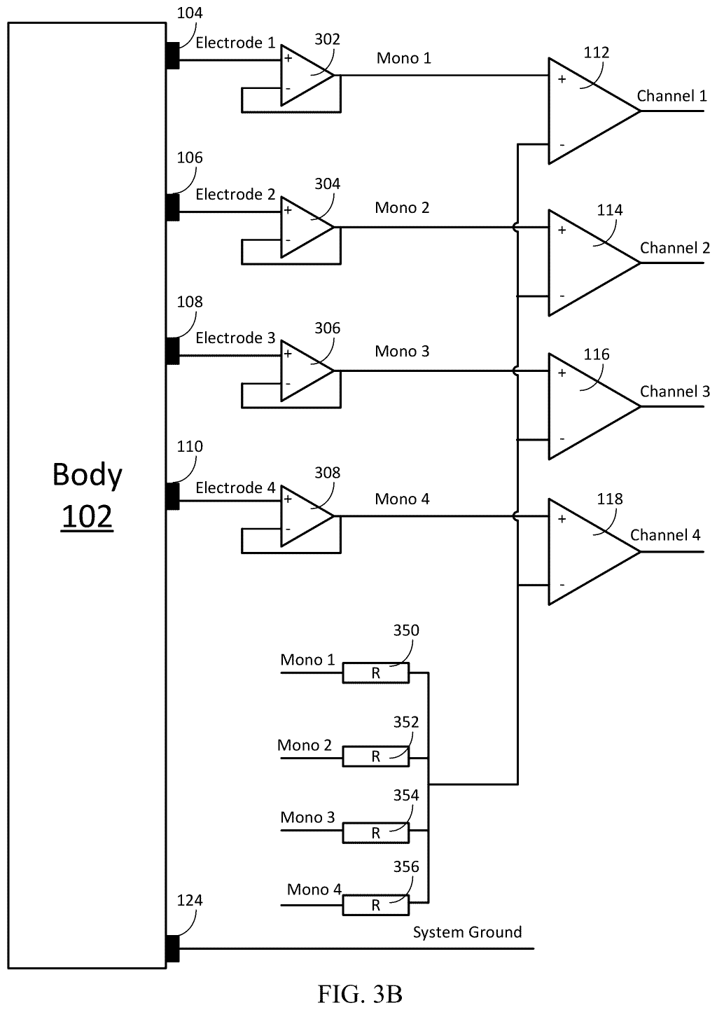

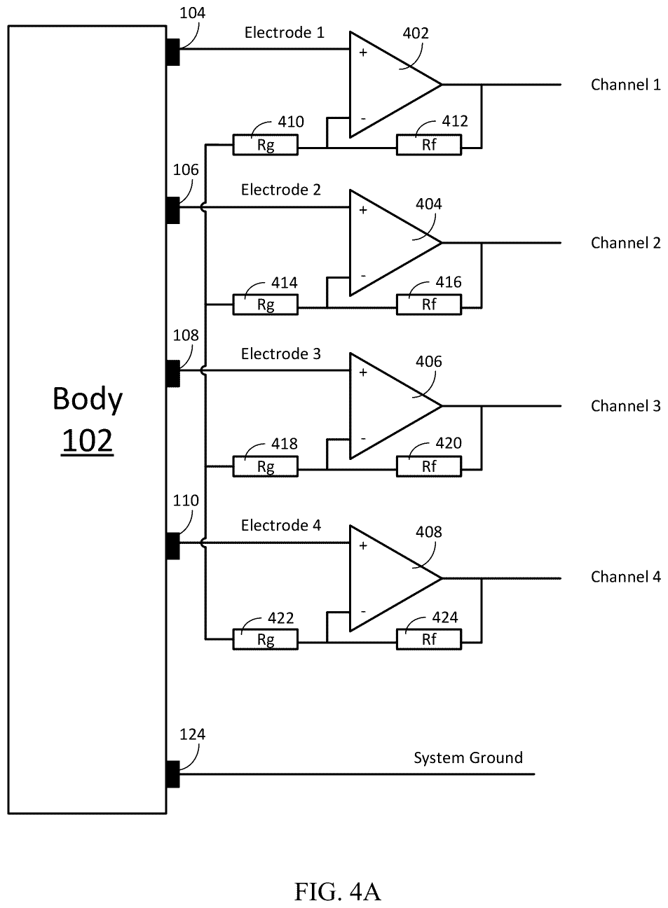

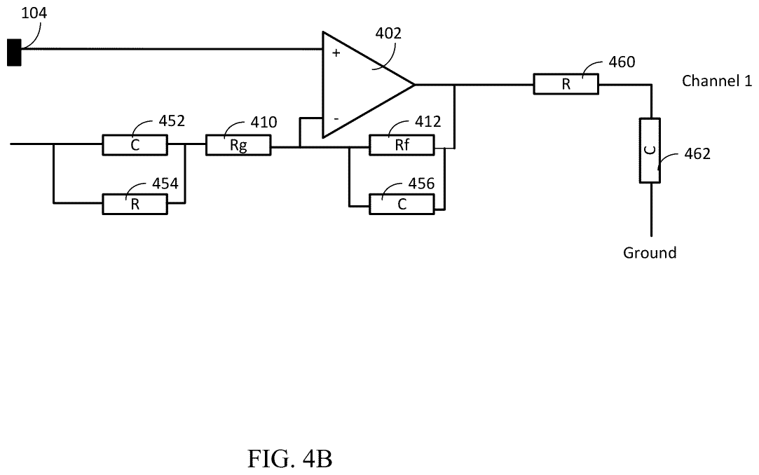

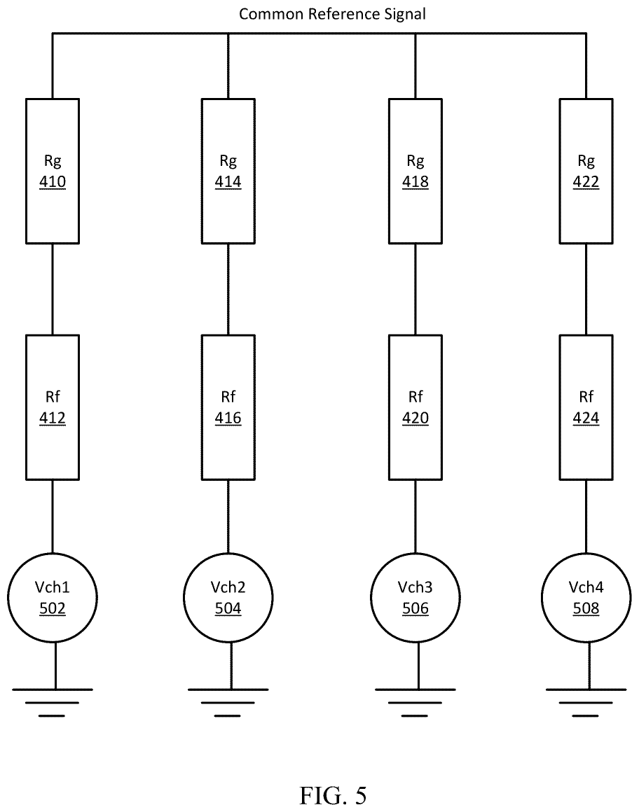

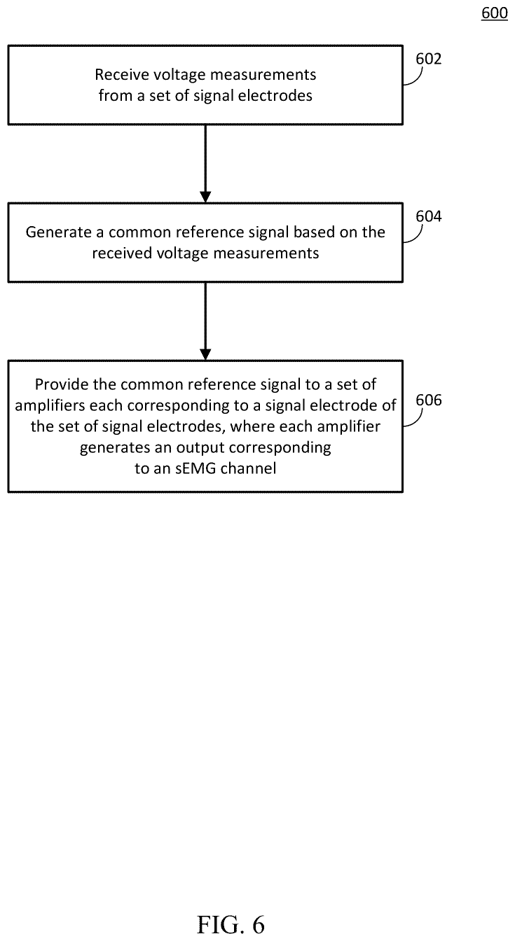

Various techniques are described to achieve pseudomonopolar configurations. In one example, a common reference electrode is used to generate a common reference signal, which is used as a comparison by multiple signal electrodes, as shown in and described below in connection with FIG. 1. In another example, signals from multiple reference electrodes are used to generate a common reference signal, which is used as a comparison by multiple signal electrodes, as shown in and described below in connection with FIG. 2. In yet another example, signal electrode outputs themselves are used to generate a common reference signal, as shown in and described below in connection with FIG. 3A. In still another example, outputs of operational amplifiers that each receive output of a signal electrode are used to generate the common reference signal, as shown in and described below in connection with FIGS. 4 and 5. In still another example, a common reference signal is generated algorithmically using outputs of signal electrodes, as shown in and described below in connection with FIG. 6.

In some embodiments, signal electrodes and/or reference electrodes may be incorporated in a wearable device, such as a wrist-worn device. For example, in some implementations, electrodes may be disposed in and/or on a portion of the wearable device such that the electrodes are each configured to be in contact with a portion of the wearer's skin. In some implementations, electrodes may be arranged circumferentially around a band of a wrist-worn device. Additionally or alternatively, in some implementations, two or more electrodes (e.g., a signal electrode and a reference electrode, two signal electrodes, etc.) may be arranged longitudinally within a portion of a band of a wrist-worn device such that the two or more electrodes are disposed along a longitudinal fiber of a muscle (e.g., as the fiber extends from elbow to wrist). In some embodiments, all electrodes may be of the same size and/or shape. Conversely, in some embodiments, different electrodes may have different sizes and/or shapes. For example, in some implementations, reference electrodes may have a different size and/or shape than signal electrodes.

It should be noted that, in instances in which one or more reference electrodes are used to generate a common reference signal, the one or more reference electrodes may measure activity from the same muscle groups as the signal electrodes. By contrast, conventional techniques for utilizing reference electrodes for measuring sEMG activity may place the electrode on an electrically neutral tissue with respect to the signal of interest. By way of example, in an instance in which the muscle activity of interest corresponds to arm, hand, wrist, and/or finger muscles, conventional techniques may place signal electrodes proximate to arm, hand, wrist, and/or finger muscles, and may place one or more reference electrodes on un-related body portions, such as the chest, the temple, the leg, or the like. In the techniques described herein, in instances in which one or more reference electrodes are used to generate the common reference signal, the reference electrodes may be positioned within proximity to the signal electrodes such that both the reference electrodes and the signal electrodes measure activity from the same muscle groups. These muscle groups may include arm, wrist, hand, and/or finger extensors, flexors, abductors, adductors, or the like. In some embodiments, reference electrodes and signal electrodes may be positioned on and/or along a wrist-worn device such that a reference electrode and a signal electrode measure activity respectively from an agonist-antagonist muscle pair, such as a wrist flexor and wrist extensor, or the like. In order to measure activity from the same muscle groups, one or more reference electrodes, if used, and the signal electrodes may all be disposed in and/or along the same wrist-worn device (e.g., along one or more band portions of the wrist-worn device, on a back portion of a capsule of the wrist-worn device, or the like). In some implementations, a reference electrode may be within proximity of the signal electrodes, e.g., within a range of about 3 millimeters-10 centimeters.

In some implementations, a common reference signal, utilized as a comparison signal for multiple signal electrodes, is generated using a single reference electrode. In other words, the single reference electrode may be utilized as a comparison by multiple signal electrodes by providing a common reference signal utilized by each of the multiple signal electrodes. For example, each signal electrode may be operatively coupled to a first input of an instrumental amplifier, where the output of each instrumental amplifier corresponds to an sEMG channel. Continuing with this example, the common reference signal, generated using the single reference electrode, may be provided to a second input (e.g., an inverting input) of the instrumental amplifier, such that the common reference signal is effectively subtracted from the signal from each signal electrode and such that, for each signal channel, the signal of the signal electrode is amplified with respect to the common reference signal. The common reference signal may be generated from a single reference electrode. Rather than being placed on an electrically neutral surface as a ground electrode would, the single reference electrode may be placed on electrically active tissue of interest, such as a portion of skin over muscle fibers of interest. As described above, the signal reference electrode may measure activity from the same muscle groups as the signal electrodes. Accordingly, the common reference signal generated by the single reference electrode may include signal of interest, such as signals from muscle fibers underlying the reference electrode. In some implementations, the output of the reference electrode may be buffered (e.g., using an operational amplifier). Buffering the output of the reference electrode may allow for impedance matching between the reference electrode and the second input of the instrumental amplifier. In other words, buffering the output of the reference electrode may allow the magnitude of the signal at the second input of each instrumental amplifier to be similar.

It should be noted that, in some implementations, multiple reference electrodes may be used such that a first reference electrode provides a common reference signal for a first set of signal electrodes, a second reference electrode provides a common reference signal for a second set of signal electrodes, and so on.

Additionally, it should be noted that, the techniques, devices, and/or systems used herein describe electrodes (signal electrodes and/or reference electrodes) that are configured to be proximate to a skin surface of a wearer of a device (e.g., a wrist-worn device, an arm-worn device, etc.). As used herein, “proximate to a skin surface” may include electrodes being in contact with the skin surface of the wearer with at least a portion of an electrode touching the skin surface of the wearer. Alternatively, “proximate to a skin surface” may include electrodes that are within a relatively small distance of the skin surface (e.g., within 1 micron, 10 microns, 100 microns, 1 millimeter, 5 millimeters, or another suitable small distance).

FIG. 1 shows a schematic diagram of an example electrode configuration that utilizes a single reference electrode to generate a common reference signal utilized to generate multiple sEMG channels. As illustrated, signal electrodes 104, 106, 108, and 110 may each be used to generate a corresponding sEMG channel (depicted as “Channel 1,” “Channel 2,” “Channel 3,” and “Channel 4,” respectively, in FIG. 1). Note that the number of signal electrodes shown in FIG. 1 is merely exemplary, and, in some embodiments, other numbers of signal electrodes (e.g., one, two, five, ten, twenty, etc.) may be used. The output of each signal electrode may be provided to a first input of a corresponding instrumental amplifier. For example, signal electrode 104 is operatively coupled to the first input of instrumental amplifier 112, signal electrode 106 is operatively coupled to the first input of instrumental amplifier 114, signal electrode 108 is operatively coupled to the first input of instrumental amplifier 116, and signal electrode 110 is operatively coupled to the first input of instrumental amplifier 118. Each signal electrode rests on a portion of body 102 of the wearer, which may be a wrist of the wearer of a wrist-worn device, the arm of a wearer of an arm-band device, the leg of the wearer of a device that is contact with a portion of the wearer's leg, etc.

The inverting input of each instrumental amplifier is operatively coupled to a reference electrode 120. As illustrated in FIG. 1, reference electrode 120 is also in contact with a portion of body 102. In other words, reference electrode 120 is not placed on electrically-neutral or electrically inactive tissue. Rather, reference electrode 120 is positioned such that reference electrode 120 measures signals from the same muscle group(s) as those measured by signal electrodes 104-110. In the example shown in FIG. 1, output of reference electrode 120 is buffered by operational amplifier 122. However, it should be noted that, in some embodiments, operational amplifier 122 may be omitted, and the output of reference electrode 120 may be directly provided to each inverting of input of instrumental amplifiers 112-118. Because each sEMG channel is a differential signal that subtracts out the common reference signal generated using reference electrode 120, artifactual signals, such as power line interference, may be removed from each sEMG channel. Moreover, because the same common reference signal is utilized to generate each sEMG channel, common mode signals of interest, such as from axially aligned muscle fibers, may be commonly incorporated in each sEMG channel.

Additionally, FIG. 1 shows a ground electrode 124, which may be used to generate the system ground.

In some implementations, a common reference signal, utilized to generate multiple sEMG channels, may be generated using multiple reference electrodes. For example, in some embodiments, the common reference signal may effectively correspond to an average of the signals of the multiple reference electrodes. In one example, outputs of each reference electrode may be buffered, for example, to provide for impedance matching across inverting inputs of multiple instrumental amplifiers to which the common reference signal is provided. Continuing with this example, the output of each buffer may then be combined via a set of resistors, where the common reference signal corresponds to the signal of the set of resistors. In some implementations, outputs of the set of resistors may be buffered via an optional operational amplifier to provide for further impedance matching. Similar to the electrode configuration shown in and described above in connection with FIG. 1, the common reference signal may then be provided to the inverting inputs of multiple instrumentational amplifier, where the output of each instrumentational amplifier is an sEMG channel.

FIG. 2 shows a schematic diagram of an example electrode configuration that utilizes multiple reference electrodes to generate a common reference signal. Similar to what is shown in and described above in connection with FIG. 1, outputs of signal electrodes 104-110 are operatively coupled to first inputs of a corresponding set of instrumental amplifiers 112-118, where the output of each instrumental amplifier corresponds to an sEMG channel. Similar to what is shown in and described above in connection with FIG. 1, the inverting input of each instrumental amplifiers receives the common reference signal. However, unlike what is shown in FIG. 1, in the electrode configuration shown in FIG. 2, the common reference signal is generated using multiple reference electrodes (e.g., reference electrodes 202, 204, 206, and 208). As illustrated in FIG. 2, the output of each reference electrode is buffered using a corresponding operational amplifier. For example, the output of reference electrode 202 is buffered using operational amplifier 210, the output of reference electrode 204 is buffered using operational amplifier 212, the output of reference electrode 206 is buffered using operational amplifier 214, and the output of reference electrode 208 is buffered using operational amplifier 216. The buffered outputs of the multiple reference electrodes are then combined via a corresponding set of resistors (e.g., resistors 218-224, as shown in FIG. 2). In some implementations, the combined signal corresponds to the common reference signal. In some embodiments, the combined signal may be optionally buffered using an optional operational amplifier 226, where the buffered combined signal corresponds to the common reference signal.

It should be noted that although FIG. 2 shows the same number of reference electrodes as signal electrodes, this is merely an example. In some implementations, a different number of reference electrodes may be used relative to the number of signal electrodes. For example, in some implementations, two reference electrodes may be used to generate a common reference signal used by eight signal electrodes. As another example, in some implementations, four reference electrodes may be used to generate a common reference signal used by ten signal electrodes. In some embodiments, multiple common reference signals may be generated, each by a different group of reference electrodes. In such embodiments, different groups of signal electrodes may utilize each common reference signal. By way of example, signal electrodes 1-4 may utilize a common reference signal generated by reference electrodes 1 and 2, and signal electrode 5-8 may utilize a common reference signal generated by reference electrodes 3 and 4. In some embodiments, signal electrode groups that utilize different common reference signals may be partially overlapping.

In some implementations, a common reference signal may be generated using signal electrodes rather than using one or more physical reference electrodes. For example, in some implementations, the common reference signal may correspond to a combination, or average, of outputs of multiple signal electrodes. As a more particular example, in some embodiments, the common reference signal may be a combination of buffered outputs of multiple signal electrodes. In some embodiments, each signal electrode may be operatively coupled to a first input of a corresponding instrumentational amplifier, and the inverting input of each instrumentational amplifier may receive the common reference signal, which is the combined output (or the combined buffered output) of at least a subset of the multiple signal electrodes. In some embodiments, the combined output (or the combined buffered output) of at least a subset of the multiple signal electrodes may in turn be buffered (e.g., to provide impedance matching, as described above) to generate the common reference signal. By generating a common reference signal without utilizing any physical reference electrodes, cost and/or space savings may be realized on a wearable device. For example, fewer total electrodes may be disposed in and/or on the wearable device. Additionally or alternatively, by not utilizing physical reference electrodes, additional signal electrodes may be utilized, thereby enabling more robust and/or more detailed sEMG acquisition.

FIG. 3A shows a schematic diagram of an example electrode configuration that generates a common reference signal using signal electrodes in accordance with some embodiments. As illustrated in FIG. 3A, outputs of signal electrodes 104-110 are buffered by corresponding operational amplifiers. For example, an output of signal electrode 104 is buffered by operational amplifier 302, an output of signal electrode 106 is buffered by operational amplifier 304, an output of signal electrode 108 is buffered by operational amplifier 306, and an output of signal electrode 110 is buffered by operational amplifier 308. The buffered outputs of each signal electrode is provided as a first input to a corresponding instrumentation amplifier (e.g., instrumentation amplifiers 112-118). The output of each instrumentation amplifier corresponds to an SEMG channel, as shown in and described above in connection with FIGS. 1 and 2.

Unlike what is shown in FIGS. 1 and 2, however, in the electrode configuration depicted in FIG. 3A, the buffered outputs of each signal electrode (depicted as “Mono 1,” “Mono 2,” “Mono 3,” and “Mono 4,” respectively), are combined via a corresponding set of resistors (e.g., resistors 310, 312, 314, and 316) to generate the common reference signal. The common reference signal is then provided to each inverting input of each instrumentation amplifier. In some implementations, the combined signal outputs may correspond to the common reference signal. In the example shown in FIG. 3A, the combined signal output is buffered by an optional operational amplifier 318, and the buffered combined signal output corresponds to the common reference signal provided to each inverting input.

It should be noted that although FIG. 3A depicts generation of the common reference signal by combining (e.g., effectively averaging) the outputs of all of the signal electrodes, in some embodiments, the common reference signal may be generated by combining a subset of outputs of the signal electrodes. For example, in an instance in which there are four signal electrodes, the common reference signal may be generated using outputs of two of the four signal electrodes or three of the four signal electrodes. Moreover, in some embodiments, multiple common reference signals may be generated, each combining outputs of at least a subset of the signal electrodes. For example, a first common reference signal may be generated using a first subset of signal electrodes (e.g., signal electrodes 1-4), and a second common reference signal may be generated using a second subset of signal electrodes (e.g., signal electrodes 5-8. In some embodiments, each subset of signal electrodes may comprise a different number of signal electrodes or the same number of signal electrodes.

FIG. 3B depicts another example implementation of an electrode configuration using a common reference signal based on one or more signal electrodes. Similar to what is shown in and described above in connection with FIG. 3A, a common reference signal is provided to each inverting input of each instrumentation amplifier based on a buffered output of each signal electrode. FIG. 3B illustrates resistors 350, 352, 354, and 356, which are placed in series with the buffered output of each signal electrode. Note that, on a printed circuit board (PCB) implementation, any of resistors 350-356 may be populated, or not, to control the number of signal electrodes that are utilized to generate the common reference signal. For example, in an instance in which only resistor 350 is populated, only the buffered output of electrode 1 is utilized to generate the common reference signal. The implementation in which only a signal resistor of 350-356 is populated, and, accordingly, the output of only a single electrode is utilized to generate the common reference signal, is sometimes referred to herein as a “single point reference,” or SPR, implementation. As another example, in an instance in which multiple of resistors 350-356 (e.g., two of the four resistors, three of the four resistors, or four of the four resistors) are populated on the PCB, the corresponding multiple buffered outputs of the corresponding multiple signal electrodes may be utilized to generate the common reference signal. The implementation in which multiple of resistors 350-356 are utilized, and accordingly, multiple signal electrodes are utilized to generate the common reference signal, is sometimes referred to herein as an input generated common reference signal.

In some implementations, a common reference signal may be generated using signal electrodes without using any physical reference electrodes, where the common reference signal is generated based on outputs of amplifiers that receive signals from the signal electrodes and generate outputs corresponding to the sEMG channels. For example, in some embodiments, an output of each signal electrode may be operatively coupled to first inputs of each of a set of operational amplifiers, where the outputs of the operational amplifier corresponds to an sEMG channel. The inverting input of each operational amplifier may receive the common reference signal, where the common reference signal is itself generated based on an output of the operational amplifier. For example, the common reference signal may represent a combination of outputs of multiple operational amplifiers. In other words, in some embodiments, the common reference signal may be generated based on a combination (e.g., effectively an average) of multiple sEMG channels (e.g., all of the sEMG channels, or a subset of the sEMG channels). By generating the common reference signal using the outputs of the operational amplifiers, less physical hardware may be required. In particular, fewer operational amplifiers may be required, because signal electrode outputs need not be buffered, as in the embodiment shown in and described above in connection with FIG. 3A. Using fewer operational amplifiers may allow for improved signal-to-noise ratio (SNR). Improved SNR may allow for overall improved performance, for example, by enabling the performance of spike sorting of individual action potential spikes, which may in turn allow for improved performance related to fine motor actions, such as typing or handwriting. Moreover, physical reference electrodes are not required, which may save space and cost and/or allow for additional signal electrodes to be used.

FIG. 4A is a schematic diagram that shows an example electrode configuration in which a common reference signal is generated using sEMG output signals in accordance with some embodiments. As illustrated, signal electrodes 104, 106, 108, and 110 are operatively coupled to first inputs of corresponding operational amplifiers 402, 404, 406, and 408. Each operational amplifier generates, as an output, a signal that corresponds to an sEMG channel. The outputs of the operational amplifiers are used to generate the common reference signal. For example, as illustrated in FIG. 4A, the outputs of the operational amplifiers may be combined via a set of resistors, such as R_{g} resistor 410, R_{f} resistor 412, R_{g} resistor 414, R_{f} resistor 416, R_{g} resistor 418, R_{f} resistor 420, R_{g} resistor 422, and R_{f} resistor 424 to generate the common reference signal. As illustrated, the common reference signal is provided at the inverting input of each operational amplifier.

FIG. 4B illustrates a schematic diagram of an example implementation of the electrode configuration shown in and described above in connection with FIG. 4A. As illustrated, capacitor 452 may be coupled in series with R_{g} resistor 410. Capacitor 452, in combination with Rg resistor 410, may act as a high pass filter to attenuate a DC offset from the sEMG signal obtained from electrode 104. The value of capacitor 452 and the value of R_{g} resistor 410 may serve to set the cut-off frequency of the high pass filter. In some implementations, capacitor 452 may have a value within a range of about 2 μF-10 μF. In some implementations R_{g} resistor 410 may have a value within a range of about 1 kohms-5 kohms. In some implementations, a resistor 454 may be placed in parallel with capacitor 452. Resistor 454 may serve as a DC path for leakage current, which may serve to stabilize the common average reference. In other words, because multiple electrodes are tied together to generate the common average reference (e.g., as shown in FIG. 4A), the common average reference may be considered weakly driven. Accordingly, by serving as a DC path for leakage current, resistor 454 may stabilize the common average reference allowing for a more robust reference signal. Note that, although capacitor 452 may allow the DC offset to be attenuated, the DC offset may not be entirely canceled. In other words, the DC information may be passed with low gain (e.g., 0 dB), which may allow the DC information to be utilized, e.g., for lead detection of individual electrodes, or the like.

As illustrated, in some embodiments, there may be a capacitor 456 in parallel with R_{f} resistor 412. Capacitor 456 and resistor 460 may together serve as a low-pass filter that performs anti-aliasing prior to providing the signal to an analog to digital converter (ADC). In some embodiments, capacitor 456 may have a value within a range of about 100 pF-300 pF. In some embodiments, R_{f} resistor 412 may have a value within a range about 200 kohms-400 kohms. In some implementations, resistor 454 may have a value within a range of about 1.5 kohms-5 kohms. As illustrated, in some implementations, there may be a capacitor 462 that connects the output to ground. Resistor 360 and capacitor 462 may serve to stabilize the input provided to the ADC. This may prevent oscillations as well as mitigate charge kickback from the ADC. In some implementations, capacitor 462 may have a value within a range of about 20 nF-50 nF.

FIG. 5 is a schematic diagram that illustrates how the circuit shown in FIG. 4A effectively averages the outputs of operational amplifiers 402, 404, 406, and 408 to generate the common reference signal. The outputs of operational amplifiers 402, 404, 406, and 408 are depicted as voltage sources 502, 504, 506, and 508, respectively. Each voltage source corresponds to an sEMG channel, as described above in connection with FIG. 4A. Resistors 410-424 are illustrated in FIG. 5. Using Millman's Theorem, the common reference signal at R_{g} 410, R_{g} 414, R_{g} 418, and R_{g} 422 may be determined by:

$$Common Reference Signal = \frac{\frac{Vch 1}{{R}_{f} + {R}_{g}} + \frac{Vch 2}{{R}_{f} + {R}_{g}} + \frac{Vch 3}{{R}_{f} + {R}_{g}} + \frac{Vch 4}{{R}_{f} + {R}_{g}}}{\frac{1}{{R}_{f} + {R}_{g}} + \frac{1}{{R}_{f} + {R}_{g}} + \frac{1}{{R}_{f} + {R}_{g}} + \frac{1}{{R}_{f} + {R}_{g}}}$$

The equation above may be reduced, such that the common reference signal may be determined by:

$$Common Reference Signal = \frac{V c h 1 + V c h 2 + V c h 3 + V c h 4}{4}$$

In other words, as illustrated by the schematic diagram shown in FIG. 5, the circuit depicted in FIG. 4A generates a common reference signal that is effectively an average (e.g., mean) of the sEMG channels. It should be noted that although FIG. 4A illustrates an electrode configuration in which the common reference signal is generated by averaging all of the sEMG channels, in some embodiments, the common reference signal may be generated by averaging a subset of the sEMG channels. For example, in some implementations, the outputs of the subset of the sEMG channels may be combined (e.g., averaged) using the technique shown in and described above in connection with FIG. 5. In some such embodiments, an inverting input of an operational amplifier may receive the common reference signal whether or not the output of the operational amplifier was used to generate the common reference signal. In some implementations, multiple common reference signals may be generated, each based on a combination (e.g., an average) of multiple sEMG channels.

In some implementations, a common reference signal may be generated digitally. For example, in some implementations, signals from a set of signal electrodes may be converted to digital signals using one or more analog-to-digital converters (ADCs). The digital signals may then be used to determine the common reference signal. For example, digital circuitry may be used to determine an average (e.g., a mean, a median, etc.) of the digital signals to generate a digital representation of the common reference signal. The digital representation of the common reference signal may then be converted to an analog common reference signal using a digital-to-analog converter (DAC). The analog common reference signal may then be provided at inverting inputs of multiple instrumentation amplifiers, each generating an output corresponding to an sEMG signal. Each instrumentation amplifier may take, at a first input, signal from a signal electrode. By way of example, referring to FIG. 3A, rather than generating the common reference signal by combining the signals from the signal electrodes using a set of resistors and an optional buffer, the signals from the signal electrodes may be provided to digital circuitry (e.g., one or more ADCs, digital circuitry to generate a digital representation of the common reference signal, and/or a DAC configured to generate an analog representation of the common reference signal).

FIG. 6 is a flowchart of an example process 600 for generating a common reference signal using digital circuitry in accordance with some embodiments. In some embodiments, blocks of process 600 may be executed by one or more processors, which may be on-board a wearable device on which or in which the signal electrodes are disposed. In some embodiments, blocks of process 600 may be executed in an order other than what is shown in FIG. 6. In some embodiments, two or more blocks of process 600 may be executed substantially in parallel. In some embodiments, one or more blocks of process 600 may be omitted.

Process 600 can begin at 602 by receiving voltage measurements from a set of signal electrodes. For example, the voltage measurements may be received by one or more ADCs operatively coupled to the signal electrodes of the set of signal electrodes. The ADCs may generate digital representations of outputs of the signal electrodes.

At 604, process 600 can generate a common reference signal based on the received voltage measurements. For example, in some embodiments, process 600 may determine an average of the received voltage measurements, where the common reference signal corresponds to the determined average. The average may be a mean, a median, or the like. The common reference signal may be generated using digital circuitry, which may include algorithms executed by one or more processors. In some implementations, the common reference signal may be a digitized version of the common reference signal. The digitized version of the common reference signal may then be converted to an analog representation using a DAC.

At 606, process 600 can provide the common reference signal to a set of amplifiers each corresponding to a signal electrode of the set of signal electrodes, where each amplifier generates an output corresponding to an sEMG channel. For example, the common reference signal may be provided to an inverting input of each amplifier, where the amplifier receives, at another input, signal from the corresponding signal electrode. The common reference signal provided to each amplifier may be an analog version of the common reference signal generated at block 604. In some embodiments, the digital circuitry that generates the common reference signal may be operatively coupled to the inverting input of each amplifier in order to provide the common reference signal to each inverting input.

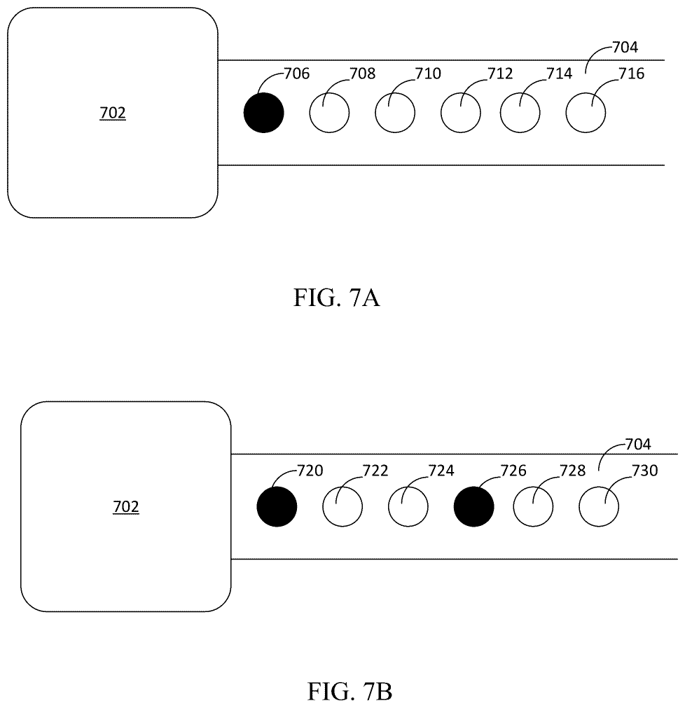

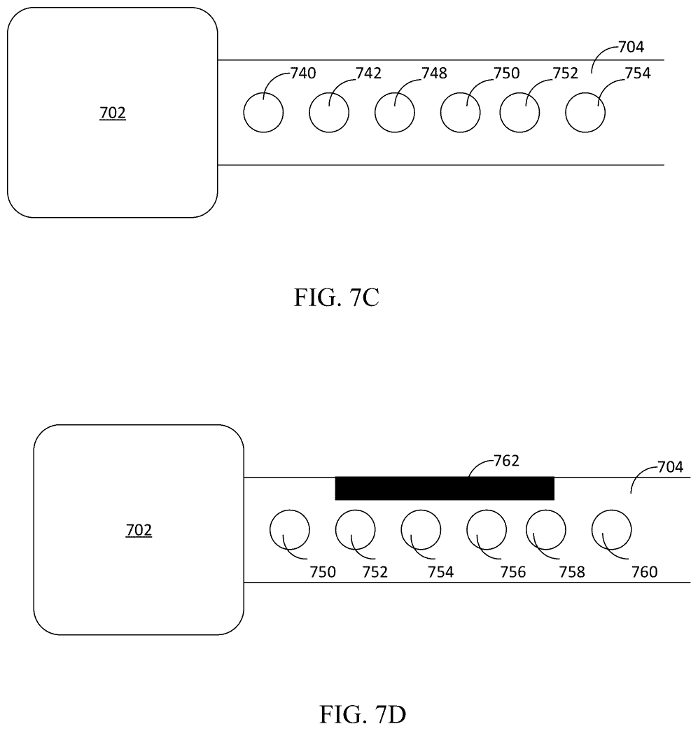

As described above, signal electrodes and reference electrodes, if used, may be disposed in, on, and/or along portions of a wearable device. FIGS. 7A-7D provide example configurations of signal electrodes and reference electrodes (if used) with respect to example wrist-worn devices. It should be noted that that examples shown in FIGS. 7A-7D may be used in connection with any suitable configurations shown in and described above in connection with FIGS. 1-6. For example, FIG. 7A, which depicts a single reference electrode, may be used to implement the configuration that utilizes a single reference electrode shown in and described above in connection with FIG. 1. Alternatively, the configuration depicted in FIG. 1 may be implemented using the single reference electrode patch shown in and described below in connection with FIG. 7D. As another example, FIG. 7B, which depicts multiple reference electrodes, may be used to implement the configuration shown in and described above in connection with FIG. 2. Alternatively, in some implementations, the reference patch depicted in FIG. 7D may be replicated to form multiple reference electrode patches, which may then be used in the configuration shown in and described above in connection with FIG. 2. As yet another example, FIG. 7C, which depicts no reference electrodes, may be used to implement the configurations depicted in FIGS. 3, 4, and/or 6, in which the common reference signal is generated without physical references.

Turning to FIG. 7A, an example wrist-worn device is depicted that utilizes a single reference electrode is shown in accordance with some embodiments. The wrist-worn device includes a capsule 702, and a band portion 704. It should be noted with respect to FIGS. 7A-7D that, in some implementations, a wrist-worn device may include two band portions (only one of which is depicted in each of FIGS. 7A-7D). In such embodiments, each band portion may include signal electrodes and/or reference electrodes (if used). Multiple electrodes are disposed along band portion 704. For example, band portion 704 includes a reference electrode 706, and signal electrodes 708, 710, 712, 714, and 716. The spacing between electrodes (e.g., an edge-to-edge spacing) may be within a range of about 3 millimeters-20 millimeters. Note that although all of the electrodes depicted in FIG. 7A are illustrated as being the same size and shape, this is merely exemplary. In some embodiments, different electrodes may be different sizes and shapes (e.g., circular, oblong, etc.). Additionally, it should be noted that although all of the electrodes are depicted as being longitudinally aligned along band portion 704, this is merely exemplary. In some implementations, two or more electrodes may be aligned along the width of band portion 704. The single reference electrode configuration depicted in FIG. 7A may be utilized to implement generation of a common reference signal using a single physical reference electrode, as shown in and described above in connection with FIG. 7A. Alternatively, in instances in which a second band portion (not shown in FIG. 7A) includes one or more reference electrodes, reference electrode 706 along with any reference electrodes disposed along the second band portion may be utilized to implement generation of a common reference signal using multiple reference electrodes, as shown in and described above in connection with FIG. 2.

Turning to FIG. 7B, an example wrist-worn device that incorporates multiple reference electrodes is shown in accordance with some embodiments. As illustrated, band portion 704 includes reference electrodes 720 and 726, and signal electrodes 722, 724, 728, and 730. Note that the spacing between reference electrodes 720 and 726 (e.g., with two signal electrodes between) is merely exemplary, and, in some embodiments, reference electrodes may be spaced apart in any suitable manner (e.g., with any suitable number of intervening signal electrodes, on opposite band portions, or the like). Moreover, it should be noted that although FIG. 7B depicts the reference electrodes as aligned with the signal electrodes along a length of band portion 704, this is merely exemplary. In some implementations, reference electrodes may be disposed width-wise along a band portion such that the reference electrode is aligned along the width of the band with a particular signal electrode. The multiple reference electrodes depicted in FIG. 7B may be utilized to generate a common reference signal using multiple reference electrodes, as shown in and described above in connection with FIG. 2.

Tuning to FIG. 7C, an example wrist-worn device that does not include physical reference electrodes is shown in accordance with some embodiments of the disclosed subject matter. As illustrated, signal electrodes 740-754 are disposed in alignment along band portion 704. Signal electrodes 740-754, or a subset of signal electrodes 740-754, may be utilized to generate the common reference signal, for example, using the techniques shown in and described above in connection with FIGS. 3-6.

Turning to FIG. 7D, an example wrist-worn device that utilizes a reference electrode patch is shown in accordance with some embodiments. As illustrated, signal electrode 750, 752, 754, 756, 758, and 760 are disposed in alignment along band portion 704. Band portion 704 also includes a reference electrode patch 762, which may be a strip along band portion 704. In some implementations, signal acquired using reference electrode patch 762 may be utilized to generate a common reference signal utilized to generate signal channels corresponding to signal electrodes 750-760, as shown in and described above in connection with FIG. 1. It should be noted that, in some implementations, a wrist-worn device may include multiple reference electrode patches. In some such embodiments, the multiple reference electrode patches may be utilized in combination to generate a common reference signal, as shown in and described above in connection with FIG. 2.

It should be noted that although the reference electrodes depicted in FIGS. 7A, 7B, and 7D are illustrated as either electrodes having the same size and shape as the signal electrodes (e.g., in the case of FIGS. 7A and 7B), or as strips (e.g., in the case of FIG. 7D), in some implementations, other reference electrode configurations may be utilized. For example, in some embodiments, a reference electrode may be a concentric ring that surrounds a signal electrode

Additionally, it should be noted that although the signal electrodes and the reference electrodes depicted in FIGS. 7A-7D are shown as aligned along a band of the wrist-worn device, in some implementations, one or more signal electrodes and/or one or more reference electrodes may be disposed along a back cover of the capsule that is configured to be in contact with the wrist of the wearer.

FIG. 4A illustrates an example implementation of an output-generated common average reference (sometimes referred to herein as “OGC”), because the common average reference signal is based on the output of one or more signal electrodes. In some instances, the OGC implementation shown in and described above in connection with FIG. 4A may have a shared reference signal that has a relatively high impedance at the node. The relatively high impedance may make the shared reference signal susceptible to noise, e.g., power line interference (PLI) noise, or the like. Moreover, in an instance in which a particular channel is anomalous (e.g., due to poor skin contact, skin conditions creating high impedance, etc.), it may be possible to remove the channel from being included in the common average reference signal, by utilizing an associated amplifier shutdown pin, however, utilizing the amplifier shutdown pin may prevent returning to use of the channel for determining the common reference signal once the channel returns to normal function.

In some embodiments, an improved shared reference may be generated for an OGC configuration. In some examples, an improved shared reference may include a buffer at an inverting input, where the buffer is configured to tap the voltage at the inverting input without affecting the R_{f} and R_{g} impedances (e.g., as shown in and described above in connection with FIG. 4A). The signal may then be passed through a resistor to meet a single node corresponding to a shared_ref signal corresponding to the common average reference signal. The shared_ref signal may then be passed through a buffer, and the output of the buffer may then be used to drive the reference point for all sensing channels in order to drive the reference point with a relatively low impedance. The relatively low impedance of the reference point node may allow the reference signal to be less susceptible to noise (e.g., PLI noise).

FIG. 9 is an example implementation of an electrode configuration for generating an improved OGC reference signal. Note that the implementation is similar to that shown in FIG. 4A, with the inclusion of a buffer that taps the inverting input at each channel amplifier. The output of each buffer (labeled as “Inv1,” “Inv2,” “Inv3,” and “Inv4”) is provided to an averaging resistor to generate an averaged signal, which is then provided to a buffer to generate the shared reference signal that drives the reference point for each channel.

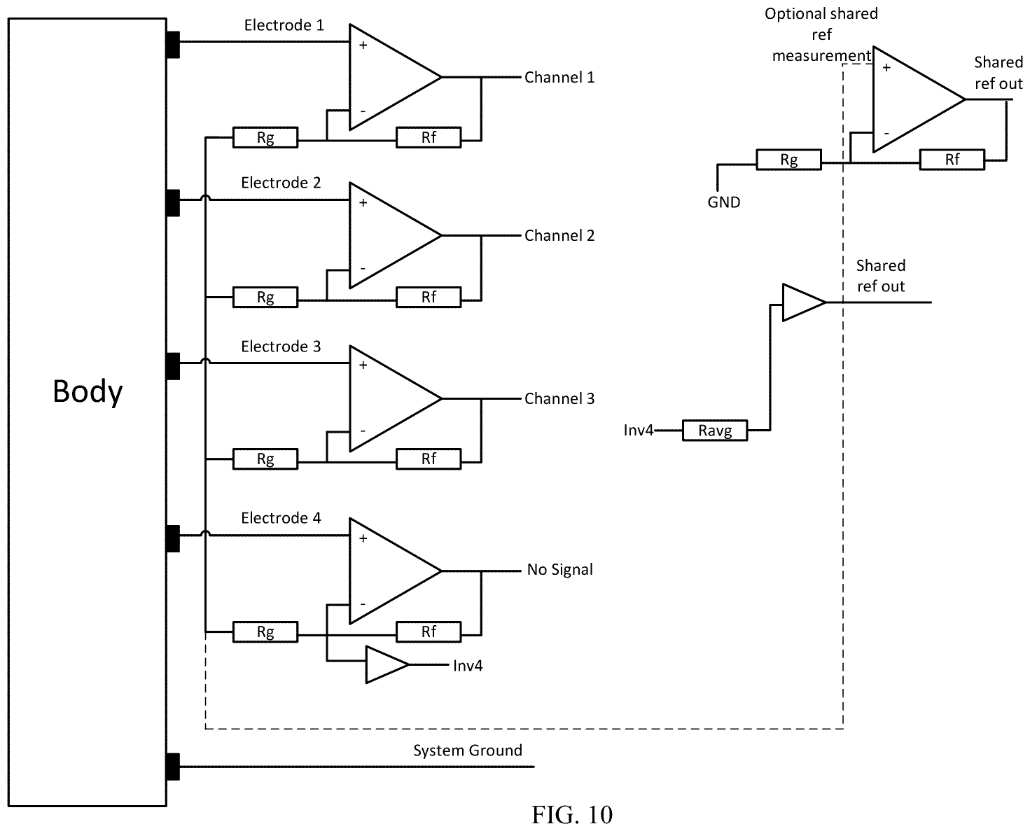

In some implementations, an OGC architecture configured to generate a common average reference signal from the output signals of one or more channels may be re-configured as a single point reference (SPR) implementation. Note that one example electrode configuration of an SPR implementation is shown in and described above in connection with FIG. 1, in which a signal from a single reference electrode is used to generate the common reference signal for multiple signal channels. However, in other embodiments, an OGC architecture may be used to generate an SPR implementation by disabling all channels other than one, which corresponds to the signal corresponding to the common reference signal. For example, referring to the OGC architecture shown in and described above in connection with FIG. 9, all buffers other than one may be disabled (e.g., via a switch), and the channel with the enabled buffer may correspond to the common reference signal. By way of example, disabling buffers associated with electrodes 1-3 and leaving the buffer associated with electrode 4 effectively yields the configuration shown in FIG. 10. In this example, the signal associated with the inverting input of the amplifier associated with electrode 4 corresponds to the common reference signal.

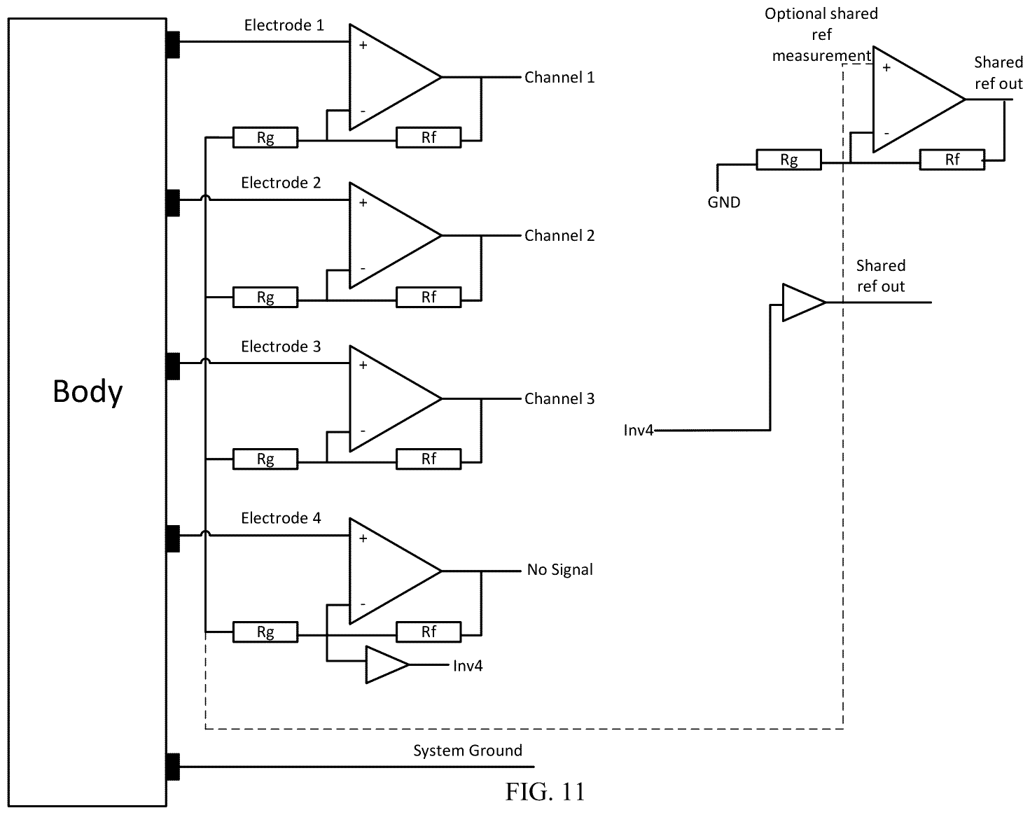

In some embodiments, the SPR implementation using OGC architecture shown in and described above in connection with FIG. 10 may be further improved to, e.g., have a larger reduction in noise. For example, a switch may be placed across the averaging resistor to short the averaging resistor. An example of the OGC architecture shown in FIG. 10 configured to implement the SPR implementation that effectively shorts the averaging resistor is shown in FIG. 11.

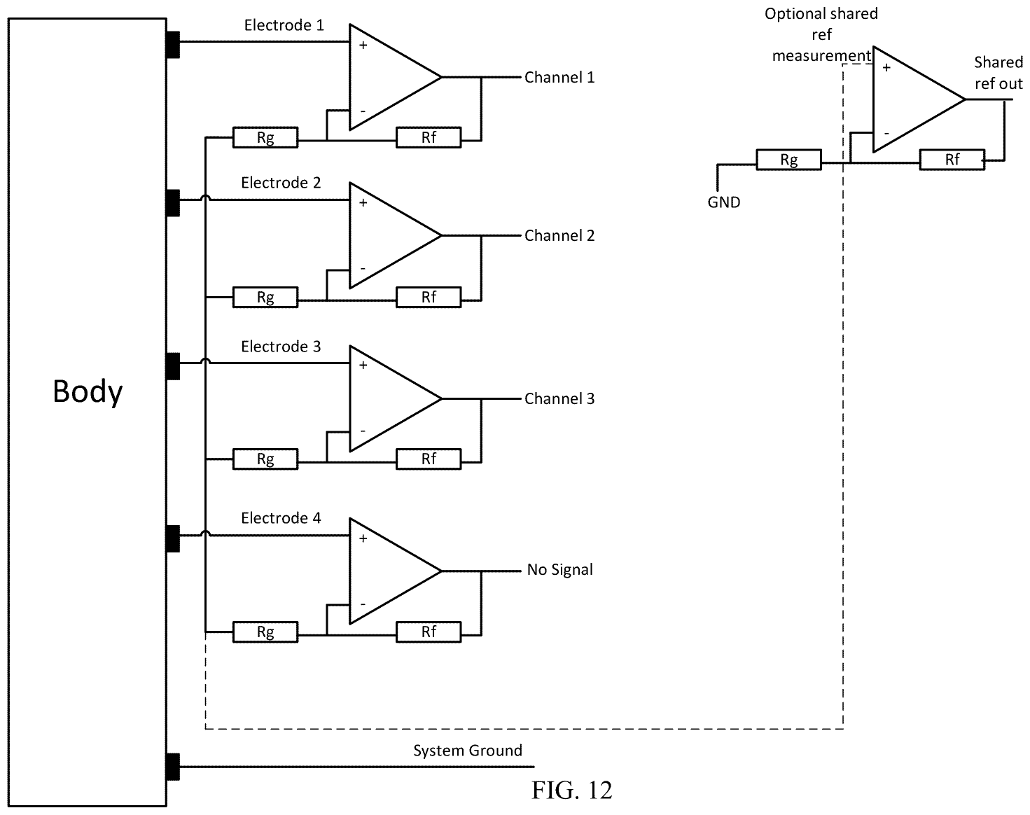

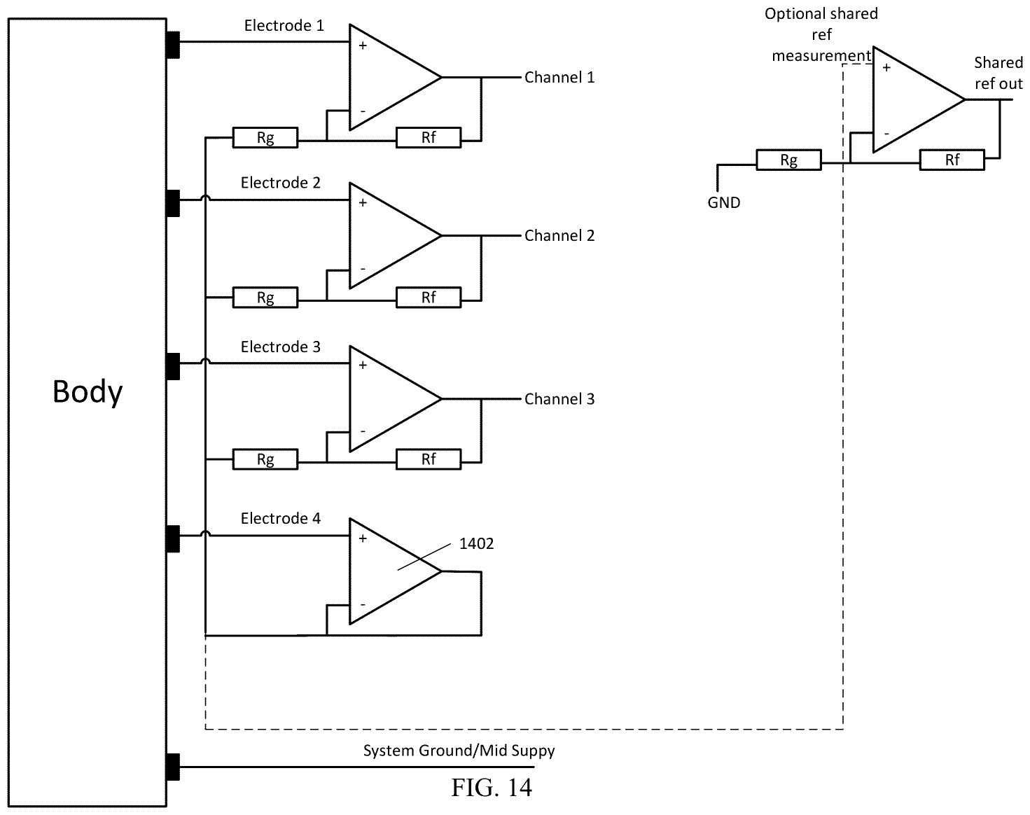

In some implementations, an OGC architecture may be used to implement an SPR implementation by shorting the R_{f} and R_{g} resistors for a channel that is to be used to provide the common reference signal. Shorting the R_{f} and R_{g} resistors for a given Electrode or channel may be accomplished by placing a switch across these resistors which may be toggled. Referring to FIG. 9, this may be implemented by disabling all buffers, and by shorting the Rf and R_{g} resistors of the channel to be used to provide the common reference signal. An example is shown in FIG. 12. In this example, the signal associated with Electrode 4 may be used as the common reference signal (note that R_{f} and R_{g} associated with Electrode 4 are shorted), and the buffers associated with Electrodes 1-3 are effectively disabled. Another example of an implementation in which an OGC architecture may be used to implement an SPR implementation is shown in FIG. 14. As illustrated, by shorting the R_{f} and R_{g} resistors associated with Electrode 4 (e.g., using switches), Electrode 4 may be used as the common reference signal). Note that, as shown in FIGS. 12 and 14, the signal associated with Electrode 4 is buffered (see buffer 1402 of FIG. 14). The output impedance of the buffer (e.g., buffer 1402) associated with the electrode being used to generate the common reference signal may be relatively low (e.g., less than about 500 ohms, less than about 300 ohms, within a range of about 100-300 ohms, or the like). The relatively low impedance may cause the signal associated with Electrode 4 to dominate the shared reference signal due to the output impedance at the buffer being less than the impedance of the R_{g} resistor. In some implementations, the output impedance of the buffer may be at least about an order of magnitude less than the output impedance of the R_{g} resistor. The configuration shown in FIGS. 12 and 14 may improve noise floor relative to a conventional SPR implementation, utilize fewer components and less board area for implementation relative to a conventional SPR implementation, and lower power consumption relative to a conventional SPR implementation.

In other words, the architecture shown in FIG. 9 may be used to flexibly and/or dynamically implement various configurations corresponding to different common reference signals. In particular, switches may be placed across one or more of: a set of R_{f} and R_{g} resistors, buffers associated with an inverting input to an sEMG channel amplifier, and/or one or more averaging resistors to selectively disable and/or short any of these circuit components. By selectively disabling and/or shorting circuit components, the OGC architecture shown in FIG. 9 may be used to implement a SPR implementation of the common reference signal. Moreover, the electrode used to generate the SPR implementation may dynamically changed, e.g., to compensate for channels with poor skin contact, etc. Alternatively, in instances in which the OGC architecture shown in FIG. 9 is used to implement an OGC reference signal, selectively disabling and/or shorting circuit components may allow for removing channels with poor skin contact or that are otherwise malfunctioning from determining of the OGC reference signal. Moreover, use of the buffers to the inverting signal may allow for a lower impedance reference signal node, thereby reducing noise interference in the common reference signal.

In some embodiments, a given electrode may be configured as either an input (e.g., an EMG signal input, an SPR reference input, or the like), or an output (e.g., a body drive, which refers to a DC drive signal, or a common mode noise cancellation (CMNC) signal). Ability to reconfigure electrodes dynamically may allow for flexibility to move sensing channels and/or body drive channels to different electrodes, e.g., responsive to poor or intermittent contact of a given electrode or complete lift off of the electrode off the skin. For example, conventionally, if a body drive channel electrode has poor or no contact, all channels may be lost due to the loss of the DC drive signal. However, using the techniques disclosed herein for dynamic electrode configuration, a different electrode with high quality contact may be reconfigured to provide the body drive signal, thereby allowing remaining channels to be used.

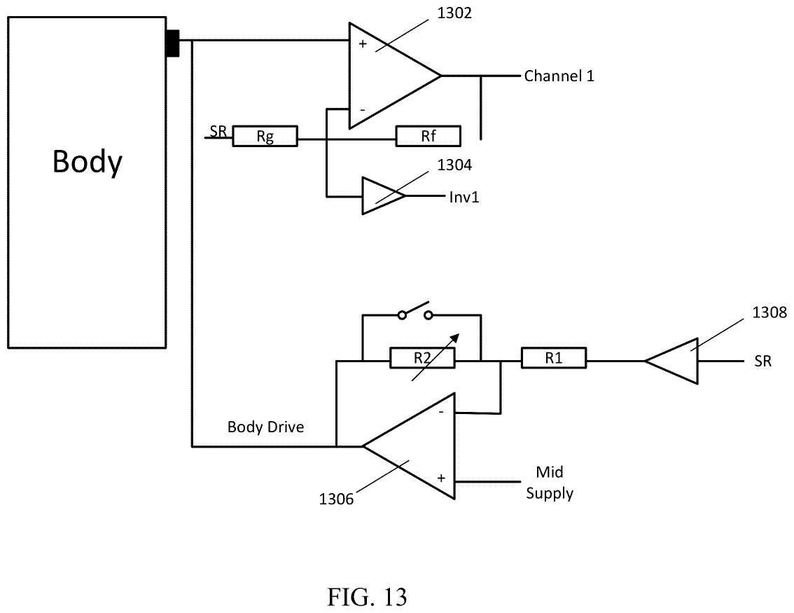

Referring to FIG. 13, electrode 1 may be configured as either an input signal (e.g., an EMG signal, and/or an SPR signal), or an output signal (e.g., a body drive signal). To configure as an output signal, EMG amplifier 1302 and/or inverting buffer 1304 may be disabled. Alternatively, to configure as an input signal, body drive amplifier 1306 and shared reference amplifier 1308 may be disabled. Note that, in some implementations, a switch between the body drive amplifier output and electrode 1 may be used if the amplifier's disabled high-impedance output significantly loads down the EMG signal.

As described above (e.g., in connection with FIGS. 12 and 14), an OGC architecture may be utilized to implement an SPR implementation. In some embodiments, rather than implementing an SPR implementation with a single electrode used to generate a common reference signal, an OGC implementation may be utilized to generate a multi-point reference (MPR) implementation. An MPR implementation may utilize multiple (e.g., two, three, five, eight, etc.) electrodes that are input to a common buffer for use in generating the common reference signal. Utilizing multiple electrodes to generate the common reference signal may aid in robustness. For instance, in implementations in which only a single electrode is used to generate the common reference signal, in an instance in which the single electrode has poor contact with the user's skin, the common reference signal may no longer be usable. In contrast, by utilizing multiple electrodes to generate the common reference signal, failure or poor contact of one electrode may be permissible, as one or more other reference electrodes may still be available to form the common reference signal.

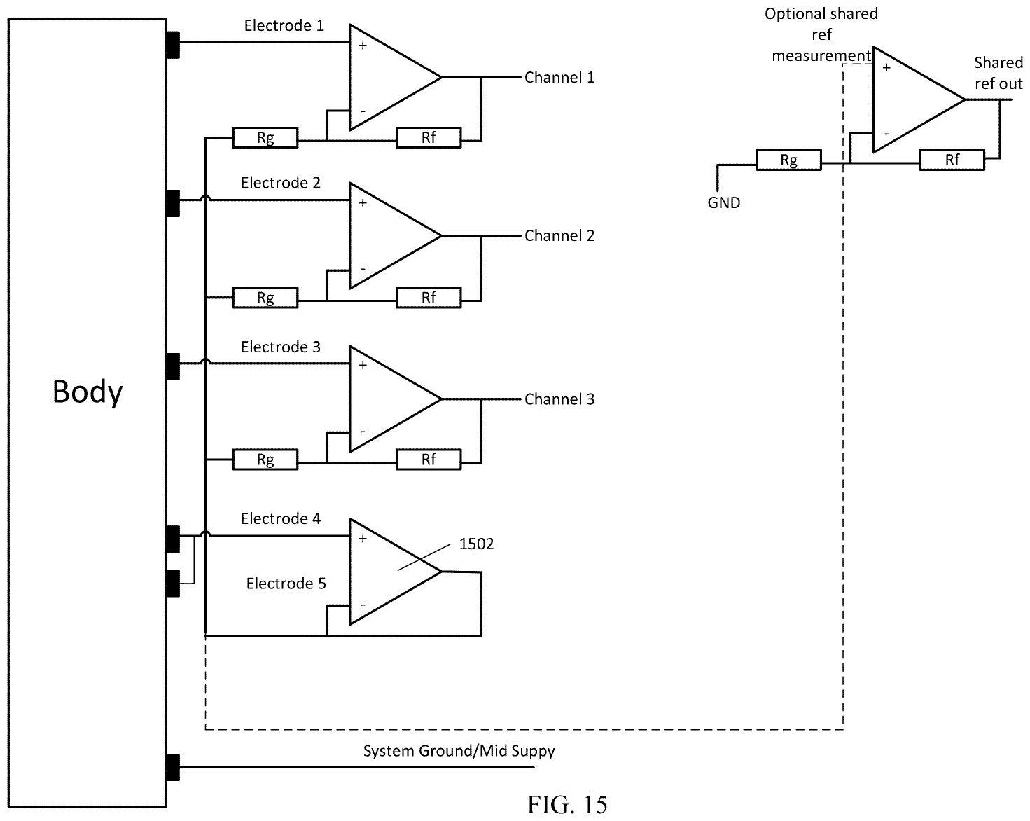

An example implementation of an MPR implementation is shown in FIG. 15. As illustrated, signals from Electrodes 4 and 5 are both provided to a buffer. The buffer 1502 may function similarly to what is described above in connection with buffer 1402 of FIG. 14 in having a relatively low output impedance (e.g., less than 500 ohms, less than 300 ohms, etc.) that allows the signals of Electrodes 4 and 5 to dominate the reference signal. Note that, although two electrodes (Electrodes 4 and 5) are shown as generating the common reference signal, in some embodiments, more electrodes may be used (e.g., three, four, five, eight, etc.) electrodes). Additionally, in some embodiments, similar to what is described above, an MPR implementation may be implemented from an OGC architecture, e.g., by use of switches that cause R_{f} and R_{g} resistors associated with electrodes to be used to form the common reference signal to be shorted.

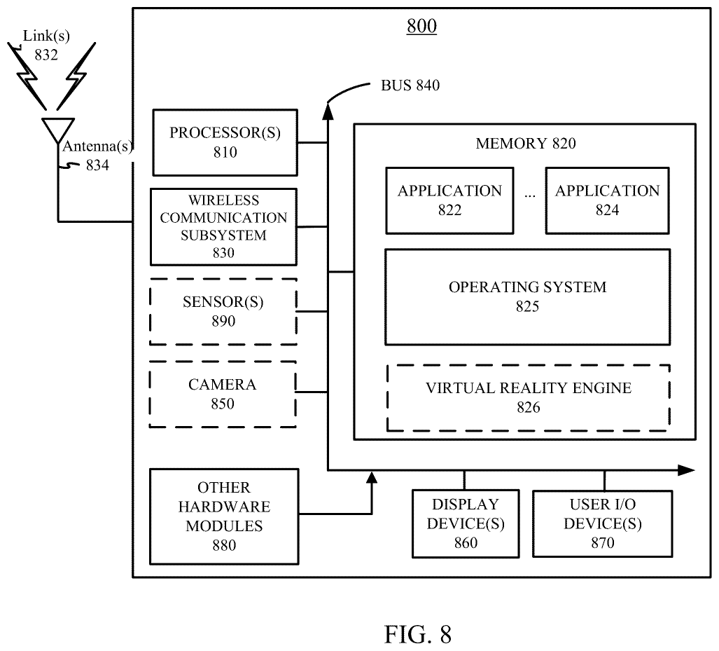

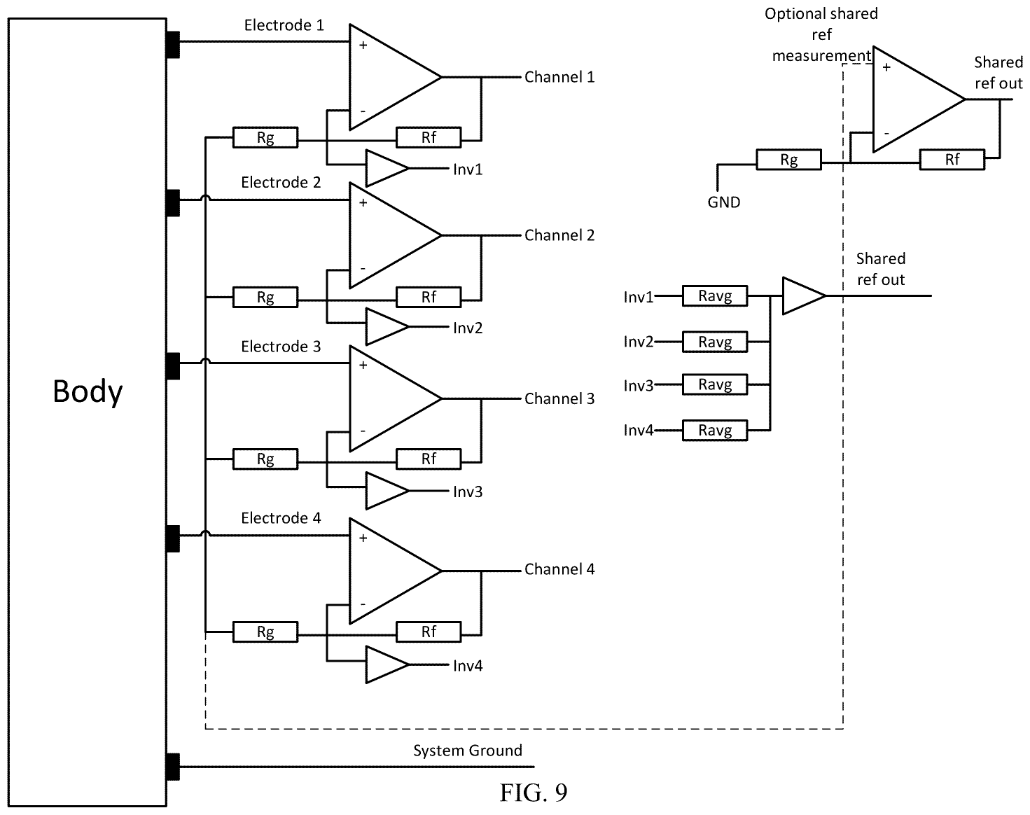

FIG. 8 is a simplified block diagram of an example of a computing system 800 for implementing some of the examples described herein. For example, in some embodiments, computing system may be used to implement a user device (e.g., a mobile phone, a tablet computer, a wrist-worn device, etc.) that implements the blocks of process 600 shown in and described above in connection with FIG. 6. In the illustrated example, computing system 800 may include one or more processor(s) 810 and a memory 820. Processor(s) 810 may be configured to execute instructions for performing operations at a number of components, and can be, for example, a general-purpose processor or microprocessor suitable for implementation within a portable electronic device. Processor(s) 810 may be communicatively coupled with a plurality of components within computing system 800. To realize this communicative coupling, processor(s) 810 may communicate with the other illustrated components across a bus 840. Bus 840 may be any subsystem adapted to transfer data within computing system 800. Bus 840 may include a plurality of computer buses and additional circuitry to transfer data.

Memory 820 may be coupled to processor(s) 810. In some embodiments, memory 820 may offer both short-term and long-term storage and may be divided into several units. Memory 820 may be volatile, such as static random access memory (SRAM) and/or dynamic random access memory (DRAM) and/or non-volatile, such as read-only memory (ROM), flash memory, and the like. Furthermore, memory 820 may include removable storage devices, such as secure digital (SD) cards. Memory 820 may provide storage of computer-readable instructions, data structures, program modules, and other data for computing system 800. In some embodiments, memory 820 may be distributed into different hardware modules. A set of instructions and/or code might be stored on memory 820. The instructions might take the form of executable code that may be executable by computing system 800, and/or might take the form of source and/or installable code, which, upon compilation and/or installation on computing system 800 (e.g., using any of a variety of generally available compilers, installation programs, compression/decompression utilities, etc.), may take the form of executable code.

In some embodiments, memory 820 may store a plurality of application modules 822 through 824, which may include any number of applications. Examples of applications may include gaming applications, conferencing applications, video playback applications, or other suitable applications. The applications may include a depth sensing function or eye tracking function. Application modules 822-824 may include particular instructions to be executed by processor(s) 810. In some embodiments, certain applications or parts of application modules 822-824 may be executable by other hardware modules 880. In certain embodiments, memory 820 may additionally include secure memory, which may include additional security controls to prevent copying or other unauthorized access to secure information.

In some embodiments, memory 820 may include an operating system 825 loaded therein. Operating system 825 may be operable to initiate the execution of the instructions provided by application modules 822-824 and/or manage other hardware modules 880 as well as interfaces with a wireless communication subsystem 830 which may include one or more wireless transceivers. Operating system 825 may be adapted to perform other operations across the components of computing system 800 including threading, resource management, data storage control and other similar functionality.

Wireless communication subsystem 830 may include, for example, an infrared communication device, a wireless communication device and/or chipset (such as a Bluetooth® device, an IEEE 802.11 device, a Wi-Fi device, a WiMax device, cellular communication facilities, etc.), and/or similar communication interfaces. Computing system 800 may include one or more antennas 834 for wireless communication as part of wireless communication subsystem 830 or as a separate component coupled to any portion of the system. Depending on desired functionality, wireless communication subsystem 830 may include separate transceivers to communicate with base transceiver stations and other wireless devices and access points, which may include communicating with different data networks and/or network types, such as wireless wide-area networks (WWANs), wireless local area networks (WLANs), or wireless personal area networks (WPANs). A WWAN may be, for example, a WiMax (IEEE 802.18) network. A WLAN may be, for example, an IEEE 802.11x network. A WPAN may be, for example, a Bluetooth network, an IEEE 802.8x, or some other types of network. The techniques described herein may also be used for any combination of WWAN, WLAN, and/or WPAN. Wireless communications subsystem 830 may permit data to be exchanged with a network, other computer systems, and/or any other devices described herein. Wireless communication subsystem 830 may include a means for transmitting or receiving data, such as identifiers of HMD devices, position data, a geographic map, a heat map, photos, or videos, using antenna(s) 834 and wireless link(s) 832. Wireless communication subsystem 830, processor(s) 810, and memory 820 may together comprise at least a part of one or more of a means for performing some functions disclosed herein.

Embodiments of computing system 800 may also include one or more sensors 890. Sensor(s) 890 may include, for example, an image sensor, an accelerometer, a pressure sensor, a temperature sensor, a proximity sensor, a magnetometer, a gyroscope, an inertial sensor (e.g., a module that combines an accelerometer and a gyroscope), an ambient light sensor, or any other similar module operable to provide sensory output and/or receive sensory input, such as a depth sensor or a position sensor. For example, in some implementations, sensor(s) 890 may include one or more inertial measurement units (IMUs) and/or one or more position sensors. An IMU may generate calibration data indicating an estimated position of a device, based on measurement signals received from one or more of the position sensors. A position sensor may generate one or more measurement signals in response to motion of a device. Examples of the position sensors may include, but are not limited to, one or more accelerometers, one or more gyroscopes, one or more magnetometers, another suitable type of sensor that detects motion, a type of sensor used for error correction of the IMU, or some combination thereof. The position sensors may be located external to the IMU, internal to the IMU, or some combination thereof. At least some sensors may use a structured light pattern for sensing.

Computing system 800 may include a display module 860. Display module 860 may be a near-eye display, and may graphically present information, such as images, videos, and various instructions, from computing system 800 to a user. Such information may be derived from one or more application modules 822-824, virtual reality engine 826, one or more other hardware modules 880, a combination thereof, or any other suitable means for resolving graphical content for the user (e.g., by operating system 825). Display module 860 may use liquid crystal display (LCD) technology, light-emitting diode (LED) technology (including, for example, OLED, ILED, μLED, AMOLED, TOLED, etc.), light emitting polymer display (LPD) technology, or some other display technology.

Computing system 800 may include a user input/output module 870. User input/output module 870 may allow a user to send action requests to computing system 800. An action request may be a request to perform a particular action. For example, an action request may be to start or end an application or to perform a particular action within the application. User input/output module 870 may include one or more input devices. Example input devices may include a touchscreen, a touch pad, microphone(s), button(s), dial(s), switch(es), a keyboard, a mouse, a game controller, or any other suitable device for receiving action requests and communicating the received action requests to computing system 800. In some embodiments, user input/output module 870 may provide haptic feedback to the user in accordance with instructions received from computing system 800. For example, the haptic feedback may be provided when an action request is received or has been performed.

Computing system 800 may include a camera 850 that may be used to take photos or videos. Camera 850 may be configured to take photos or videos of the user. Camera 850 may also be used to take photos or videos of the environment, for example, for VR, AR, or MR applications. Camera 850 may include, for example, a complementary metal-oxide-semiconductor (CMOS) image sensor with a few millions or tens of millions of pixels. In some implementations, camera 850 may include two or more cameras that may be used to capture 3-D images.

In some embodiments, computing system 800 may include a plurality of other hardware modules 880. Each of other hardware modules 880 may be a physical module within computing system 800. While each of other hardware modules 880 may be permanently configured as a structure, some of other hardware modules 880 may be temporarily configured to perform specific functions or temporarily activated. Examples of other hardware modules 880 may include, for example, an audio output and/or input module (e.g., a microphone or speaker), a near field communication (NFC) module, a rechargeable battery, a battery management system, a wired/wireless battery charging system, etc. In some embodiments, one or more functions of other hardware modules 880 may be implemented in software.

In some embodiments, memory 820 of computing system 800 may also store a virtual reality engine 826. Virtual reality engine 826 may execute applications within computing system Y100 and receive position information, acceleration information, velocity information, predicted future positions, or some combination thereof from the various sensors. In some embodiments, the information received by virtual reality engine 826 may be used for producing a signal (e.g., display instructions) to display module 860. For example, if the received information indicates that the user has looked to the left, virtual reality engine 826 may generate content that mirrors the user's movement in a virtual environment. Additionally, virtual reality engine 826 may perform an action within an application in response to an action request received from user input/output module 870 and provide feedback to the user. The provided feedback may be visual, audible, or haptic feedback. In some implementations, processor(s) 810 may include one or more GPUs that may execute virtual reality engine 826.

In various implementations, the above-described hardware and modules may be implemented on a single device or on multiple devices that can communicate with one another using wired or wireless connections. For example, in some implementations, some components or modules, such as GPUs, virtual reality engine 826, and applications (e.g., tracking application), may be implemented on two or more paired or connected devices.

In alternative configurations, different and/or additional components may be included in computing system 800. Similarly, functionality of one or more of the components can be distributed among the components in a manner different from the manner described above. For example, in some embodiments, computing system 800 may be modified to include other system environments, such as an AR system environment and/or an MR environment.

Embodiments disclosed herein may be used to implement components of an artificial reality system or may be implemented in conjunction with an artificial reality system. Artificial reality is a form of reality that has been adjusted in some manner before presentation to a user, which may include, for example, a virtual reality, an augmented reality, a mixed reality, a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, for example, create content in an artificial reality and/or are otherwise used in (e.g., perform activities in) an artificial reality. The artificial reality system that provides the artificial reality content may be implemented on various platforms, including an HMD connected to a host computer system, a standalone HMD, a mobile device or computing system, or any other hardware platform capable of providing artificial reality content to one or more viewers.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

Specific details are given in the description to provide a thorough understanding of the embodiments. However, embodiments may be practiced without these specific details. For example, well-known circuits, processes, systems, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing various embodiments. Various changes may be made in the function and arrangement of elements without departing from the spirit and scope of the present disclosure.

Also, some embodiments were described as processes depicted as flow diagrams or block diagrams. Although each may describe the operations as a sequential process, many of the operations may be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It will be apparent to those skilled in the art that substantial variations may be made in accordance with specific requirements. For example, customized or special-purpose hardware might also be used, and/or particular elements might be implemented in hardware, software (including portable software, such as applets, etc.), or both. Further, connection to other computing devices such as network input/output devices may be employed.

With reference to the appended figures, components that can include memory can include non-transitory machine-readable media. The term “machine-readable medium” and “computer-readable medium” may refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including, but not limited to, non-volatile media, volatile media, and transmission media. Common forms of computer-readable media include, for example, magnetic and/or optical media such as compact disk (CD) or digital versatile disk (DVD), punch cards, paper tape, any other physical medium with patterns of holes, a RAM, a programmable read-only memory (PROM), an erasable programmable read-only memory (EPROM), a FLASH-EPROM, any other memory chip or cartridge, a carrier wave as described hereinafter, or any other medium from which a computer can read instructions and/or code. A computer program product may include code and/or machine-executable instructions that may represent a procedure, a function, a subprogram, a program, a routine, an application (App), a subroutine, a module, a software package, a class, or any combination of instructions, data structures, or program statements.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that are also expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, etc.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific embodiments have been described, these are not intended to be limiting. Various modifications and equivalents are within the scope of the following claims.

What is claimed is:

1. A device for sensing neuromuscular signals, the device comprising:
a wearable structure configured to be worn by a user;
a plurality of signal electrodes aligned along an interior portion of the wearable structure configured to be proximate to a skin surface of the user when the device is donned by the user, each signal electrode of the plurality of signal electrodes being configured to detect neuromuscular signals of the user;
a plurality of amplifiers corresponding to the plurality of signal electrodes, wherein each amplifier includes (i) a first input operatively coupled to a corresponding signal electrode of the plurality of signals electrodes, (ii) an inverting input, and (iii) an output corresponding to a neuromuscular signal channel; and
one or more buffers configured to tap a voltage at the inverting input of a respective amplifier of the plurality of amplifiers; and
circuitry configured to operatively couple a plurality of outputs of the plurality of amplifiers to generate a common mode reference signal, wherein the common mode reference signal is provided to the inverting input of one or more amplifiers of the plurality of amplifiers.

2. The device of claim 1, further comprising a switch disposed across each buffer of the one or more buffers.

3. The device of claim 2, wherein a switch associated with a first buffer of the one or more buffers causes the first buffer to be enabled, and wherein switches associated with the remaining buffers of the one or more buffers cause the remaining buffers to be disabled.

4. The device of claim 1, wherein the circuitry comprises a set of resistors operatively coupled to output of the one or more buffers configured to generate an average of the outputs of the one or more buffers.

5. The device of claim 4, further comprising a switch disposed across each resistor of the set of resistors.

6. The device of claim 5, the switch may be utilized to short each resistor of the set of resistors.

7. The device of claim 1, wherein a given output of a given amplifier of the plurality of amplifiers is operatively coupled to the inverting input via one or more resistors.

8. The device of claim 7, further comprising switches disposed across at least one of the one or more resistors, wherein a given switch is configured to cause the at least one of the one or more resistors to be shorted.

9. The device of claim 1, wherein the wearable structure comprises a wrist-worn structure.

10. The device of claim 1, wherein the plurality of signal electrodes are disposed circumferentially around the interior portion of the wearable structure.

11. The device of claim 1, wherein the neuromuscular signals are associated with wrist extensor muscles and/or wrist flexor muscles.

12. A device for sensing neuromuscular signals, the device comprising:
a wearable structure configured to be worn by a user;
a plurality of signal electrodes aligned along an interior portion of the wearable structure configured to be proximate to a skin surface of the user, each signal electrode of the plurality of signal electrodes configured to detect neuromuscular signals;
a plurality of amplifiers corresponding to the plurality of signal electrodes, wherein an amplifier of the plurality of amplifiers has: a first input operatively coupled to a corresponding signal electrode of the plurality of signals electrodes; an inverting input; and an output corresponding to a neuromuscular signal channel; and
circuitry configured to operatively couple a plurality of outputs of the plurality of amplifiers and a buffered output of at least one reference electrode to generate a common mode reference signal, wherein the common mode reference signal is provided to the inverting input of each amplifier of the plurality of amplifiers.

13. The device of claim 12, wherein the at least one reference electrode is selected from the plurality of signal electrodes.

14. The device of claim 13, wherein the at least one reference electrode is selected from the plurality of signal electrodes by shorting one or more resistors that operatively couple the amplifier associated with the at least one reference electrode to the inverting input.

15. The device of claim 12, wherein the at least one reference electrode comprises two or more reference electrodes.

16. The device of claim 12, further comprising a buffer that receives signal from the at least one reference electrode and generates the buffered output.

17. The device of claim 16, wherein an output impedance of the buffer is less than about 300 ohms.

18. The device of claim 12, wherein the wearable structure comprises a wrist-worn structure.

19. The device of claim 12, wherein the plurality of signal electrodes are disposed circumferentially around the interior portion of the wearable structure.

20. The device of claim 12, wherein the neuromuscular signals are associated with wrist extensor muscles and/or wrist flexor muscles.
